(19) United States
(12) Patent Application Publication
(10) Pub. No.: US 2026/0061142 A1
(43) Pub. Date: Mar. 5, 2026

(54) MULTI-CHANNEL INFUSION MICRONEEDLE

(71) Applicants: GEORGIA TECH RESEARCH CORPORATION, Atlanta, GA (US); BOARD OF REGENTS FOR THE OKLAHOMA AGRICULTURAL AND MECHANICAL COLLEGES, Stillwater, OK (US)

(72) Inventors: Yue CHEN, Atlanta, GA (US); Anthony GUNDERMAN, Atlanta, GA (US); Rudy MONTAYRE, Atlanta, GA (US); Ashish RANJAN, Stillwater, OK (US); Akansha SINGH, Stillwater, OK (US)

(21) Appl. No.: 19/106,580

(22) PCT Filed: Aug. 28, 2023
(86) PCT No.: PCT/US2023/031292

§ 371 Date: Feb. 25, 2025

Related U.S. Application Data
(60) Provisional application No. 63/401,296, filed on Aug. 26, 2022.

(51) Int. Cl. A61M 5/32; A61M 5/315

(52) U.S. Cl. CPC A61M 5/3297; A61M 5/31526; A61M 2005/3201

(57) ABSTRACT

Provided is a fluid delivery device for delivering at least two fluids, the device comprising: a first fluid pathway having a first upstream large volume and a first downstream small volume connected in fluid communication with the first upstream large volume, the first fluid pathway further comprising a first exit; a second fluid pathway having an second upstream large volume and a downstream second small volume connected in fluid communication with the second upstream large volume, the second fluid pathway further comprising a second exit; and an insertion member containing the first and second exits in a closely proximate relationship and configured to insert the first and second exits through tissue to a distribution location without prior mixing of the fluids. Also provided is a kit containing the fluid delivery device, methods of using the fluid delivery device, and methods of manufacturing the fluid delivery device.

### CROSS-REFERENCE TO RELATED APPLICATIONS

This application claims priority to, and the benefit of, U.S. Provisional Patent Application No. 63/401,296 , filed Aug. 26, 2022, entitled “BI-CHANNEL INFUSION MICRONEEDLE,” which is incorporated by reference herein in its entirety.

### BACKGROUND

Injection or administration of fluids is routine practice in fields like medicine and scientific research. Often, more than one fluid needs to be injected or administered. This is commonly performed by injecting each fluid subsequently using separate fluid delivery devices. Though the fluids could potentially be mixed together, loaded into and injected using the same injecting device, this premixing may reduce some effectiveness of the treatment.

Thus, there is a need for a fluid delivery device which can inject more than one fluid, and which can do so without the fluids mixing prior to injection. These needs and others are at least partially satisfied by the present disclosure.

### SUMMARY

In an embodiment, provided is a fluid delivery device for delivering at least two fluids, the device comprising: a first fluid pathway having a first upstream large volume and a first downstream small volume connected in fluid communication with the first upstream large volume, wherein a diameter of the pathway decreases from the first upstream large volume to the first downstream small volume, the first fluid pathway further comprising a first exit having an exit diameter smaller than a storage diameter of the first upstream large volume; a second fluid pathway having an second upstream large volume and a downstream second small volume connected in fluid communication with the second upstream large volume, wherein a diameter of the pathway decreases from the second upstream large volume to the downstream small volume, the second fluid pathway further comprising a second exit having an exit diameter smaller than a storage diameter of the first upstream large volume; and an insertion member containing the first and second exits in a closely proximate relationship and configured to insert the first and second exits through tissue to a distribution location without prior mixing of the fluids.

In another embodiment, provided is a method of simultaneously administering two or more fluids using any of the disclosed fluid delivery devices. In another embodiment, provided is a method of treating a subject in need, the method comprising injecting two different fluids into the subject using any of the disclosed fluid delivery devices.

In another embodiment, provided is a kit, comprising a fluid delivery device, wherein the fluid delivery device comprises: a first syringe configured to receive a first fluid; a second syringe configured to receive a second fluid; a coupling comprising a channel guide separator coupled to a syringe tip; and a support comprising a syringe coupling and a plunger coupling; wherein the channel guide separator comprises a first channel and a second channel; wherein the first syringe is configured to be coupled to the first channel and the second syringe is configured to be coupled to the second channel; and wherein the support is configured to be coupled to both of the first syringe and the second syringe.

In another embodiment, provided is a method of manufacturing a fluid delivery device, the method comprising: a) additively manufacturing a channel guide separator; b) fabricating a first inner cannula; and c) injecting a first sealant into the channel guide separator around the first inner cannula.

In another aspect, provided is a linear rail assembly for manufacturing a fluid delivery device, comprising: a base comprising a rail; a slide movably coupled to the rail; an injector comprising a body configured to receive a viscous fluid, an injection tip, and a plunger adjacent to the slide; and a torque mechanism for moving the slide along the rail; wherein a torque force required to operate the torque mechanism is less than a normal force required to depress the plunger.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIG. 1 depicts a perspective view of an exemplary fluid delivery device.

FIG. 2 depicts a top view of the partially disassembled exemplary fluid delivery device of FIG. 1.

FIG. 3 depicts another perspective view of the exemplary fluid delivery device of FIG. 1.

FIG. 4 depicts a top view of the exemplary fluid delivery device of FIG. 1 in addition to a zoomed in view of the needle portion.

FIG. 5 depicts a partial medial cross section of a top view of the exemplary fluid delivery device of FIG. 1, as well as a zoomed in medial cross section of the needle portion and a zoomed in partial medial cross section of the channel guide separator of said exemplary fluid delivery device.

FIG. 6 depicts a top view of the exemplary fluid delivery device of FIG. 1 showing a sealant injection port.

FIG. 7 depicts a perspective view of another exemplary fluid delivery device, as well as a zoomed in view of the needle portion of said exemplary fluid delivery device.

FIG. 8 depicts a top view of the exemplary fluid delivery device of FIG. 7, as well as a zoomed in partial medial cross section of the channel guide separator of said exemplary fluid delivery device.

FIG. 9 depicts an axial cross section of the needle portion of the exemplary fluid delivery device of FIG. 7.

FIGS. 10A-10B depict a top view of a channel guide separator (FIG. 10A) and a modified channel guide separator (FIG. 10B) of the exemplary fluid delivery device of FIG. 7. The coupling mechanism is not shown for clarity.

FIG. 11 depicts an exemplary disassembled fluid delivery device.

FIG. 12 depicts exemplary parts of a disassembled fluid delivery device.

FIG. 13 depicts another exemplary disassembled fluid delivery device.

FIG. 14 depicts an exemplary assembled fluid delivery device.

FIG. 15 depicts an exemplary plunger coupling and syringe coupling in further detail.

FIG. 16 depicts a side view of an exemplary fluid delivery device.

FIG. 17 depicts step 1 in assembling an exemplary fluid delivery device.

FIG. 18 depicts step 2 in assembling an exemplary fluid delivery device.

FIG. 19 depicts step 3 in assembling an exemplary fluid delivery device.

FIG. 20 depicts step 4 in assembling an exemplary fluid delivery device.

FIG. 21 depicts step 5 in assembling an exemplary fluid delivery device.

FIG. 22 depicts step 6 in assembling an exemplary fluid delivery device.

FIG. 23 depicts step 7 in assembling an exemplary fluid delivery device.

FIG. 24 depicts an exemplary means of disassembling the syringe coupling of a fluid delivery device.

FIG. 25 depicts an exemplary linear rail assembly.

FIGS. 26A-26B depict exemplary results for the ablation of male rat tissues (right and left testicles) using an exemplary fluid delivery device to determine feasibility. FIG. 26A shows out of the six male rats treated, four showed complete remission of the testes within two months of a single injection. Two rats exhibited a partial response, yet all subjects demonstrated a significant and consistent reduction in treated volumes over the four-month observation period post-injection. FIG. 26B shows by the conclusion of four months, the fully ablated tissues had undergone resorption, with only fatty tissues remaining (indicated by the red ellipse). On the other hand, the partially ablated testes presented a fibrotic tissue texture (highlighted by the blue ellipse), underscoring the high efficacy of this treatment injection device.

The components in the drawings are not necessarily to scale relative to each other. Like reference, numerals designate corresponding parts throughout the several views.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### DEFINITIONS

In this specification and in the claims that follow, reference will be made to a number of terms, which shall be defined to have the following meanings:

Throughout the description and claims of this specification, the word “comprise” and other forms of the word, such as “comprising” and “comprises,” means including but not limited to, and are not intended to exclude, for example, other additives, segments, integers, or steps. Furthermore, it is to be understood that the terms comprise, comprising, and comprises as they relate to various aspects, elements, and features of the disclosed invention also include the more limited aspects of “consisting essentially of” and “consisting of.”

As used herein, the singular forms “a,” “an,” and “the” include plural referents unless the context clearly dictates otherwise. Thus, for example, reference to a “polymer” includes aspects having two or more such polymers unless the context clearly indicates otherwise.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

For the terms “for example” and “such as,” and grammatical equivalences thereof, the phrase “and without limitation” is understood to follow unless explicitly stated otherwise.

### EXAMPLE DEVICE AND METHOD OF MANUFACTURE

In an embodiment, provided is a fluid delivery device for delivering at least two fluids, the device comprising: a first fluid pathway having a first upstream large volume and a first downstream small volume connected in fluid communication with the first upstream large volume, wherein a diameter of the pathway decreases from the first upstream large volume to the first downstream small volume, the first fluid pathway further comprising a first exit having an exit diameter smaller than a storage diameter of the first upstream large volume; a second fluid pathway having an second upstream large volume and a downstream second small volume connected in fluid communication with the second upstream large volume, wherein a diameter of the pathway decreases from the second upstream large volume to the downstream small volume, the second fluid pathway further comprising a second exit having an exit diameter smaller than a storage diameter of the first upstream large volume; and an insertion member containing the first and second exits in a closely proximate relationship and configured to insert the first and second exits through tissue to a distribution location without prior mixing of the fluids. As used herein, the term “at least two fluids” refers to any two or more fluids which can have different chemical compositions, different concentrations, different physical properties, or which are the same but are contained within two or more separate vessels.

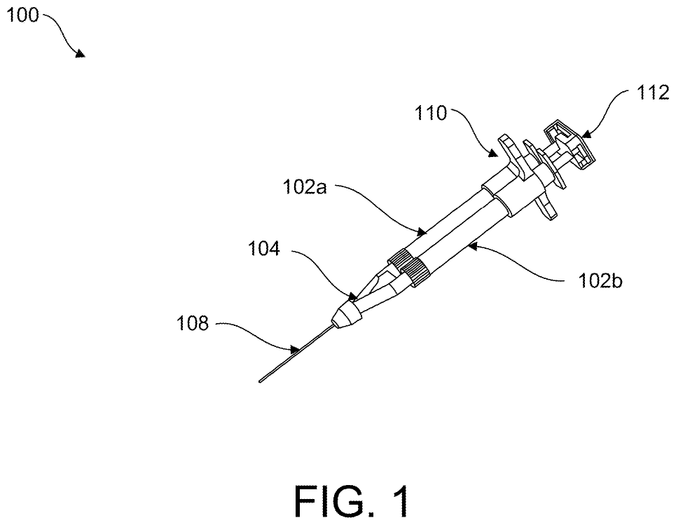

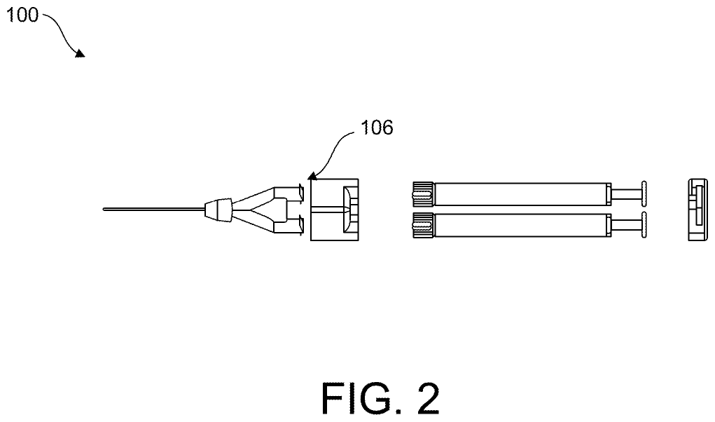

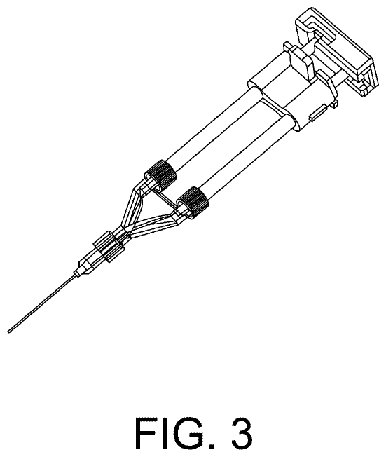

An example fluid delivery device 100 is shown in FIG. 1, FIG. 2, and FIG. 3. The exemplary fluid delivery device has two syringes 102a and 102b which serve as the first and second upstream large volumes, respectively. The first syringe 102a has a proximal end and a distal end, includes a first plunger, and is configured to receive a first fluid. The second syringe 102b has a proximal end and a distal end, includes a second plunger, and is configured to receive a second fluid. In some embodiments, the syringes 102a and 102b are sterile. In some embodiments, the syringes 102a and 102b are not sterile.

To support the two syringes 102a and 102b, a syringe coupling 110 surrounds the distal end of both of the two syringes 102a and 102b. A plunger coupling 112 is added over the plungers of both of the two syringes 102a and 102b to allow simultaneous injection from both syringes. In some embodiments, the syringe coupling 110 is manufactured by injection molding, additive manufacturing, compression molding, thermoforming, or other suitable manufacturing techniques. In some embodiments, the syringe coupling 110 is removably coupled to the two syringes 102a and 102b. In some embodiments, the syringe coupling 110 can be reused. In some embodiments, the syringe coupling 110 is fixedly couped to the two syringes 102a and 102b. In some embodiments, the plunger coupling 112 is manufactured by injection molding, additive manufacturing, compression molding, thermoforming, or other suitable manufacturing techniques. In some embodiments, the plunger coupling 112 is removably coupled to the two syringes 102a and 102b. In some embodiments, the plunger coupling 112 can be reused. In some embodiments, the plunger coupling 112 is fixedly couped to the two syringes 102a and 102b.

The syringes 102a and 102b are coupled to a proximal end of a channel guide separator 104 via a luer lock 106. In other embodiments, the syringes 102a and 102b are coupled to a proximal end of the channel guide separator via screw threads, a snap coupling, a sliding coupling, or other suitable coupling mechanisms. A distal end of the channel guide separator 104 is further coupled to a syringe tip 108, which serves as the insertion member. In some embodiments, the channel guide separator 104 is sterile. In some embodiments, the channel guide separator 104 is not sterile. In some embodiments, the syringe tip 108 is sterile. In some embodiments, the syringe tip 108 is not sterile.

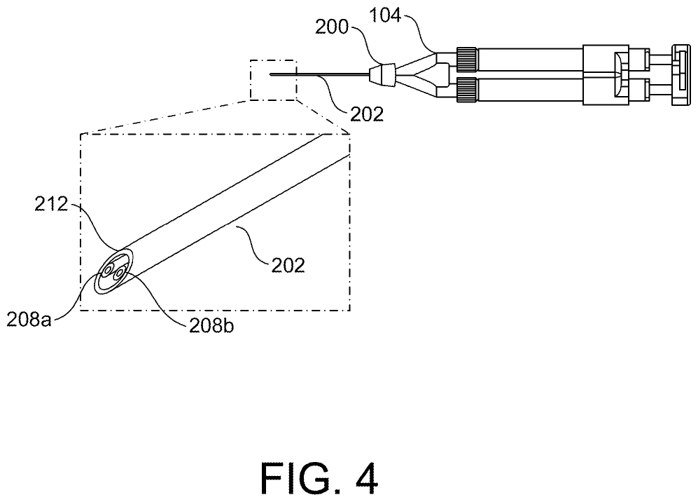

FIG. 4 shows the syringe tip 108 and the channel guide separator 104 in more detail. The syringe tip 108 includes a coupling mechanism 200 and a needle portion 202. The needle portion 202 includes a main cannula 212. In some embodiments, the main cannula 212 has a needle gauge from about 8 G to about 34 G, including exemplary values of about 9 G, about 10 G, about 11 G, about 12 G, about 13 G, about 14 G, about 15 G, about 16 G, about 17 G, about 18 G, about 19 G, about 20 G, about 21 G, about 22 G, about 23 G, about 24 G, about 25 G, about 26 G, about 27 G, about 28 G, about 29 G, about 30 G, about 31 G, about 32 G, and about 33 G. In some embodiments, the syringe tip 108 is a custom manufactured syringe needle. In some embodiments, the syringe tip 108 is a commercially available syringe needle. In some embodiments, the syringe tip 108 is a pipette tip.

The channel guide separator 104 includes a first channel 204a, a second channel 204b, and a central exit port 206. In some embodiments, the first channel 204a has a same volume and/or shape as the second channel 204b. In some embodiments, the first channel 204a has a different volume and/or shape than the second channel 204b. The proximal end of the first syringe 102a is coupled to the first channel 204a and the proximal end of the second syringe 102 is coupled to the second channel 204b. In some embodiments, each of the first syringe 102a and the second syringe 102b is coupled to each of the first channel 204a and the second channel 204b via a luer lock, screw threads, a snap coupling, a sliding coupling, or other suitable coupling mechanisms. A distal end of the first channel 204a and a distal end of the second channel 204b merge into the central exit port 206. In some embodiments, the channel guide separator 104 does not include a central exit port 206. A distal end of the central exit port 106 is coupled to a proximal end of the needle portion 202 via the coupling mechanism 200. In some embodiments, the coupling mechanism 200 is a luer lock, screw threads, a snap coupling, a sliding coupling, or other suitable coupling mechanisms.

The channel guide separator further includes a first inner cannula 208a and a second inner cannula 208b, which serve as the first and second exits, respectively. In some embodiments, each of the first inner cannula 208a and the second inner cannula 208b has a needle gauge from about 8 G to about 34 G, including exemplary values of about 9 G, about 10 G, about 11 G, about 12 G, about 13 G, about 14 G, about 15 G, about 16 G, about 17 G, about 18 G, about 19 G, about 20 G, about 21 G, about 22 G, about 23 G, about 24 G, about 25 G, about 26 G, about 27 G, about 28 G, about 29 G, about 30 G, about 31 G, about 32 G, and about 33 G. In some embodiments, the first inner cannula 208a has a different needle gauge than the second inner cannula 208b. In some embodiments, the first inner cannula 208a has the same needle gauge as the second inner cannula 208b. In some embodiments, each of the first inner cannula 208a and the second inner cannula 208b has a smaller needle gauge than the main cannula 212. In some embodiments, the channel guide separator includes more than two inner cannulas.

A proximal end of the first inner cannula 208a is in the first channel 204a and a distal end of the first inner cannula 208a is in a distal end of the main cannula 212. A proximal end of the second inner cannula 208b is in the second channel 204b and a distal end of the second inner cannula 208b is in a distal end of the main cannula 212. The distal end of the first inner cannula 208a is adjacent to the distal end of the second inner cannula 208a. In some embodiments, each of the first inner cannula 208a and the second inner cannula 208b has a smaller diameter than each of the first inner cannula 204a and the second inner cannula 204b.

Each of the first inner cannula 208a and the second inner cannula 208b is contained by the first channel 204a, the central exit port 206, and the main cannula 212. In some embodiments, the first inner cannula 208a and the second inner cannula 208b are separated in the needle portion 202 by a distance that is from about 0.1 mm to about 20 mm, including exemplary values of about 0.2 mm, about 0.4 mm, about 0.6 mm, about 0.8 mm, about 1 mm, about 2 mm, about 3 mm, about 4 mm, about 5 mm, about 6 mm, about 7 mm, about 8 mm, about 9 mm, about 10 mm, about 12 mm, about 14 mm, about 16 mm, and about 18 mm. In some embodiments, the first inner cannula 208a is immediately adjacent to the second inner cannula 208b in the needle portion 202. At the end of the needle portion 202, the main cannula 212 extends slightly beyond the two inner cannulas 208a and 208b to prevent damage to the ends of the two inner cannulas. In some embodiments, the first inner cannula 208a and 208b are the same length. In some embodiments, the first inner cannula 208a is a different length than the second inner cannula 208b. In some embodiments, the central cannula extends from about 0.1 mm to about 20 mm beyond the two inner cannulas, including exemplary values of about 0.2 mm, about 0.4 mm, about 0.6 mm, about 0.8 mm, about 1 mm, about 2 mm, about 3 mm, about 4 mm, about 5 mm, about 6 mm, about 7 mm, about 8 mm, about 9 mm, about 10 mm, about 12 mm, about 14 mm, about 16 mm, and about 18 mm. In some embodiments, the main cannula 212 and each of the two inner cannulas 208a and 208b are the same length.

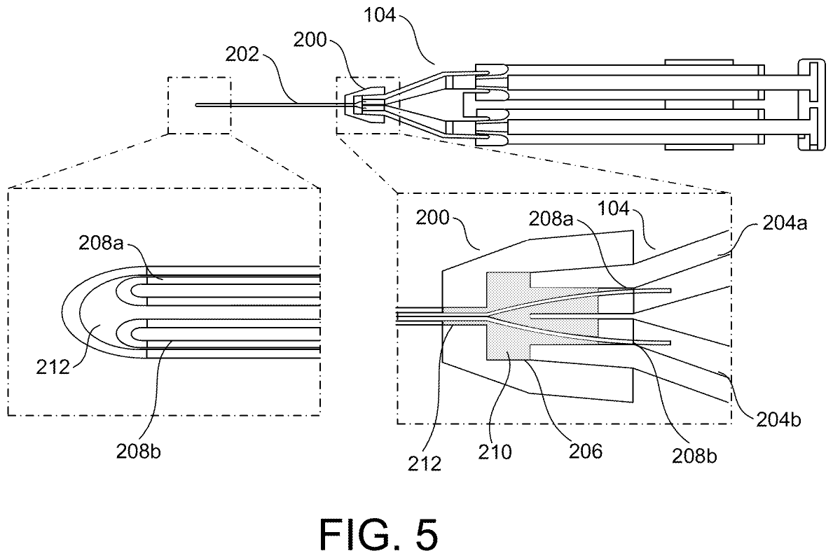

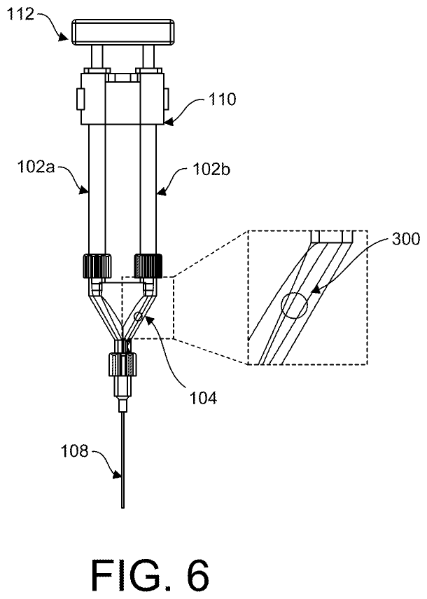

The channel guide separator 104 further includes a sealant 210 shown in gray in FIG. 5. The sealant 210 surrounds the first inner cannula 208a and the second inner cannula 208b and fills the central exit port 206. The sealant 210 creates a barrier to the central exit port 206. In some embodiments, the sealant 210 is epoxy, polyester resin, polyurethane resin, acrylic resin, latex resin, silicone resin, or other suitable sealants or combinations thereof. In some embodiments, sealant 210 does not fill the central exit port 206 and includes a first sealant that surrounds the first inner cannula 208a and a second sealant that surrounds the second inner cannula 208b. In some embodiments, the first sealant and the second sealant are different materials. In some embodiments, the first sealant and the second sealant are the same material. In some embodiments, the first sealant is present in the same amount as the second sealant. In some embodiments, the first sealant is present in a different amount than the second sealant. The sealant 210 configures exclusively the first inner cannula 208a to receive the first fluid and exclusively the second inner cannula 208b to receive the second fluid. In some embodiments, the first fluid and the second fluid do not intermix while contained within any part of the fluid delivery device. In some embodiments, the channel guide separator 104 further includes at least one sealant injection port 300, an exemplary version of which is shown in FIG. 6. In some embodiments, the channel guide separator 104 does not include a sealant injection port.

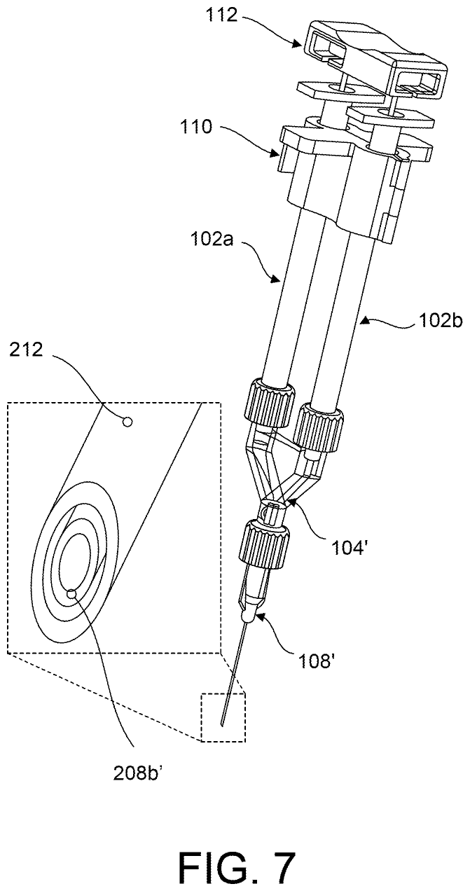

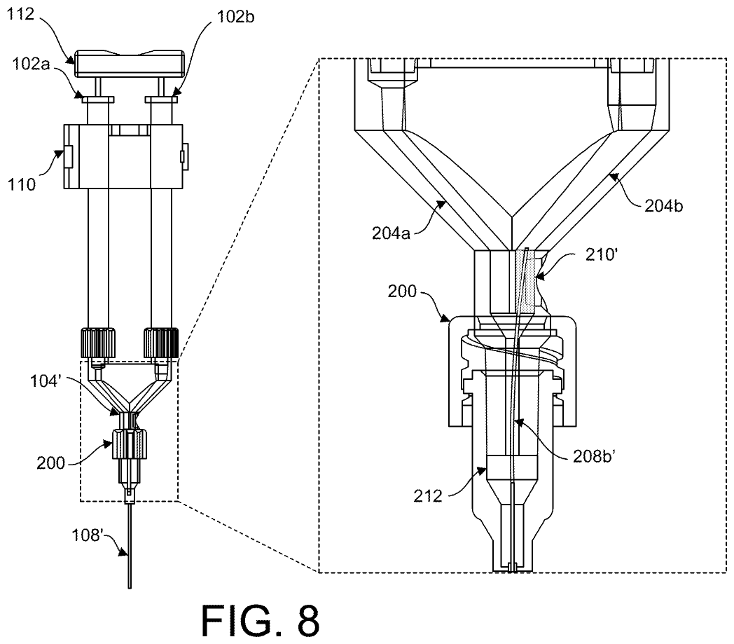

FIG. 7 and FIG. 8 show the example fluid delivery device 100 with a modified channel guide separator 104′and a modified syringe tip 108′. The channel guide separator 104′ includes the first channel 204a and the second channel 204b which merge into the central exit port 206. The channel guide separator 104′ further includes an inner cannula 208b′ which serves as the first exit. The syringe tip 108′ includes the coupling mechanism 200 and a needle portion 202′. The needle portion 202′ includes the main cannula 212 which serves as the second exit. A proximal end of the inner cannula 208b′ is in the second channel 204b and a distal end of the inner cannula 208b′ is in a distal end of the main cannula 212.

In some embodiments, the inner cannula 208b′ has a needle gauge from about 8 G to about 34 G, including exemplary values of about 9 G, about 10 G, about 11 G, about 12 G, about 13 G, about 14 G, about 15 G, about 16 G, about 17 G, about 18 G, about 19 G, about 20 G, about 21 G, about 22 G, about 23 G, about 24 G, about 25 G, about 26 G, about 27 G, about 28 G, about 29 G, about 30 G, about 31 G, about 32 G, and about 33 G. In some embodiments, the inner cannula 208b′ has a smaller needle gauge than the main cannula 212. The inner cannula 208b′ is encompassed by the second channel 204b, the central exit port 206, and the main cannula 212. At the end of the needle portion 202′, the main cannula 212 extends slightly beyond the inner cannula 208b′ to prevent damage to the ends of the inner cannula. In some embodiments, the central cannula extends from about 0.1 mm to about 20 mm beyond the two inner cannulas, including exemplary values of about 0.2 mm, about 0.4 mm, about 0.6 mm, about 0.8 mm, about 1 mm, about 2 mm, about 3 mm, about 4 mm, about 5 mm, about 6 mm, about 7 mm, about 8 mm, about 9 mm, about 10 mm, about 12 mm, about 14 mm, about 16 mm, and about 18 mm. In some embodiments, the main cannula 212 and the inner cannula 208b′ are the same length.

The channel guide separator 104′ further includes a sealant 210′. The sealant 210′ surrounds the inner cannula 208b′. The sealant 210′ creates a barrier to the central exit port 206. In some embodiments, the sealant 210′ is epoxy, polyester resin, polyurethane resin, acrylic resin, latex resin, silicone resin, or other suitable sealants or combinations thereof. The sealant 210′ configures exclusively the inner cannula 208b′ to receive the first fluid and exclusively the central exit port 206 and the main cannula 212 to receive the second fluid. In some embodiments, the first fluid and the second fluid do not intermix while contained within any part of the fluid delivery device. In some embodiments, the channel guide separator 104′ further includes at least one sealant injection port. In some embodiments, the channel guide separator 104′ does not include a sealant injection port.

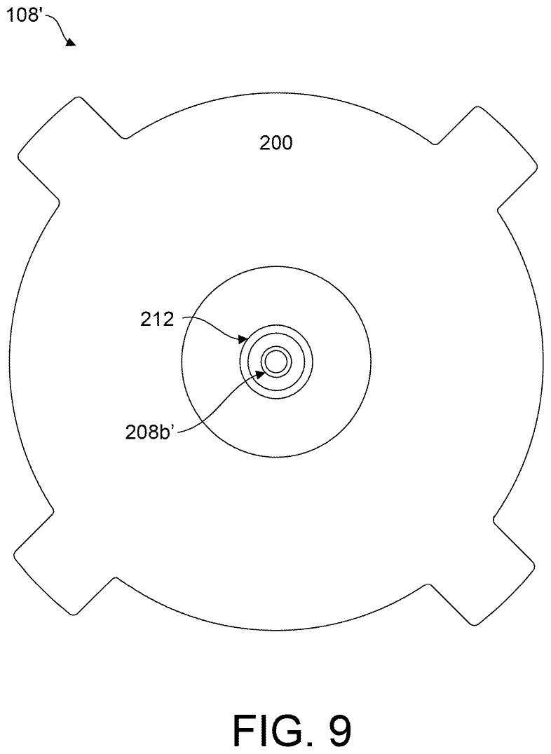

FIG. 9 shows a cross section of the syringe tip 108′. The coupling mechanism surrounds the main cannula 212, and the main cannula surrounds the inner cannula 208b′. The main cannula 212 is configured to receive the first fluid and the inner cannula 208b′ is configured to receive the second fluid (i.e., the first fluid and the second fluid travel along the needle portion 108′ concentrically).

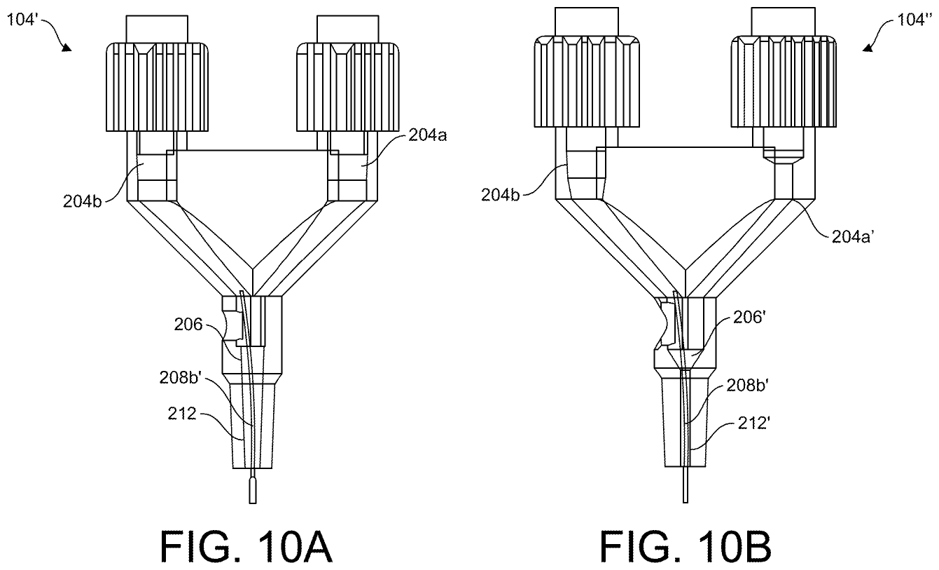

FIG. 10A shows the channel guide separator 104′, and FIG. 10B shows a modified embodiment of the channel guide separator 104″ with the coupling mechanism 200 not shown for clarity. In some embodiments, the first fluid pathway has a first volume and the second fluid pathway has a second volume. In some embodiments, the first volume is different than the second volume. In some embodiments, the first volume is the same as the second volume. In some embodiments, the volume of the first fluid pathway can be made numerically the same as the volume of the second fluid pathway via dead volume optimization to form the modified channel guide separator 104″. The modified channel guide separator 104″ has a 204a', an exit port 206′, and a main cannula 212′that have been modified in size and shape to make the volume of the first fluid pathway the same as the volume of the second fluid pathway.

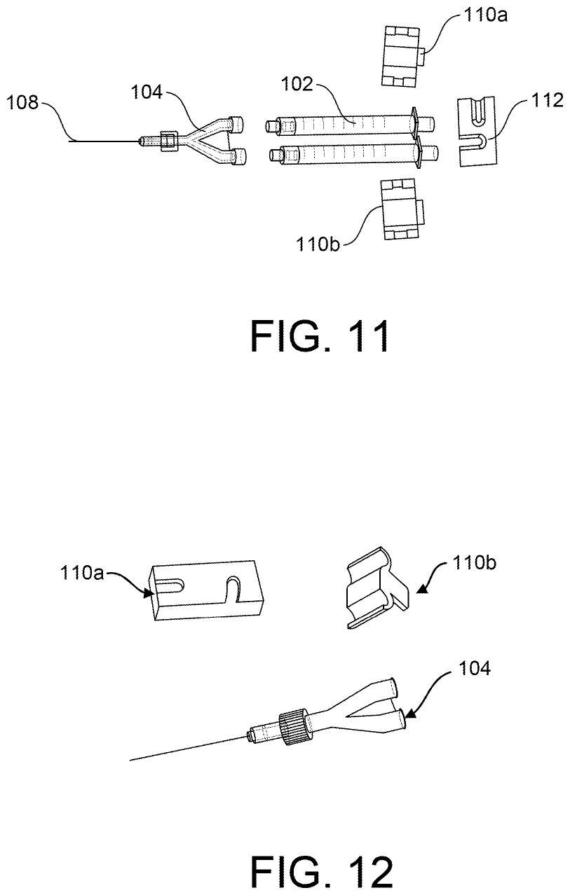

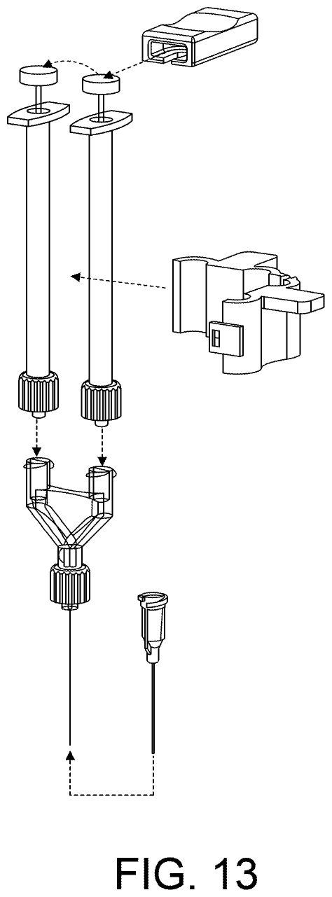

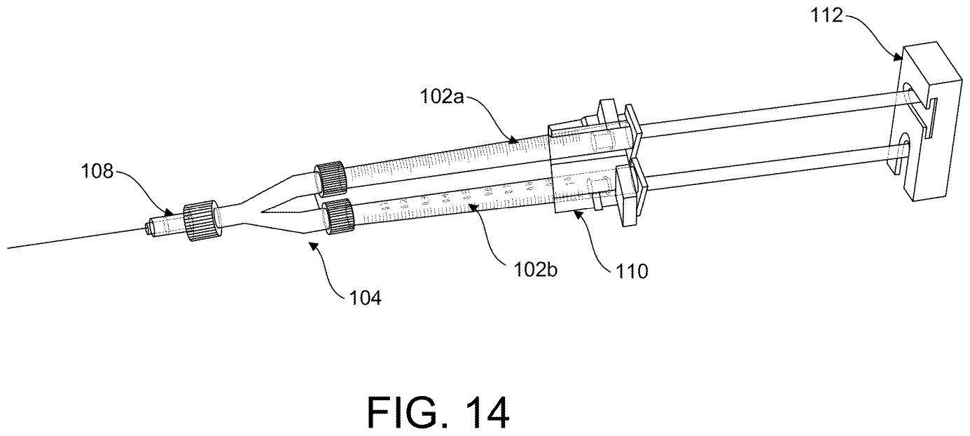

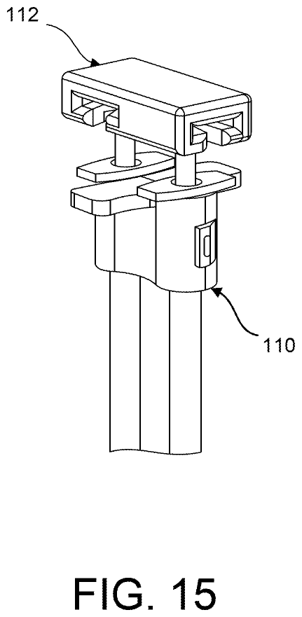

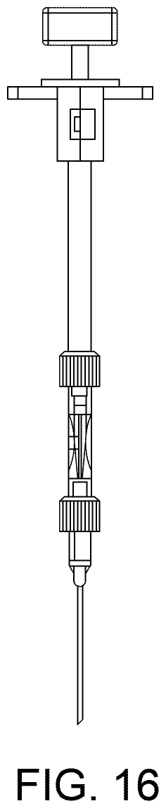

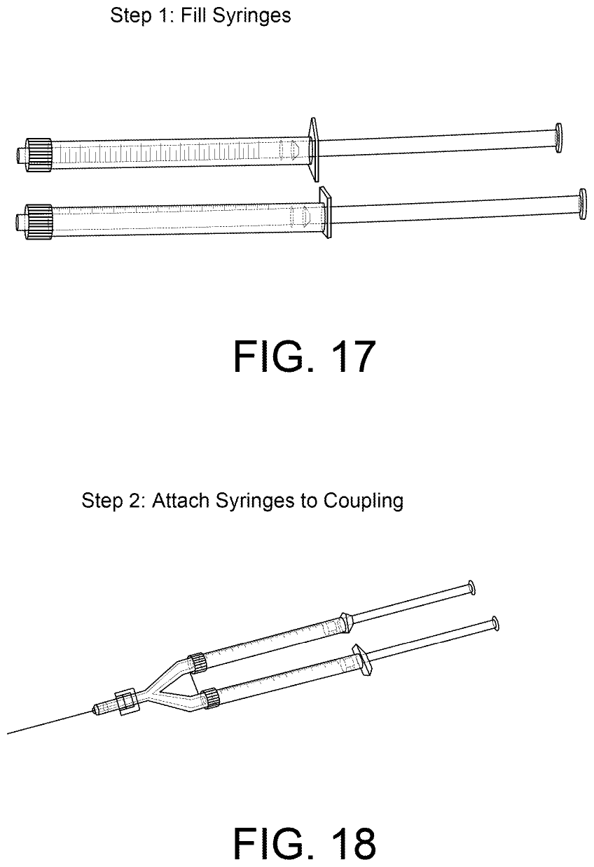

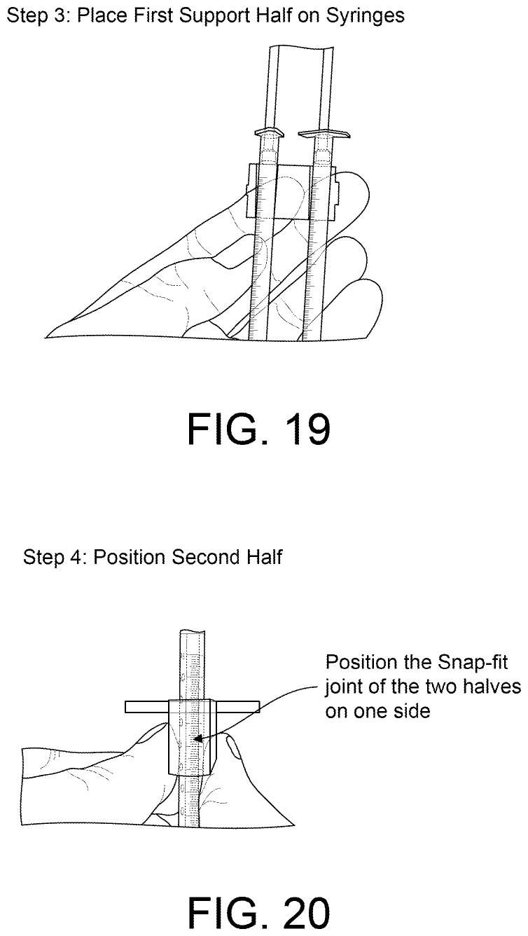

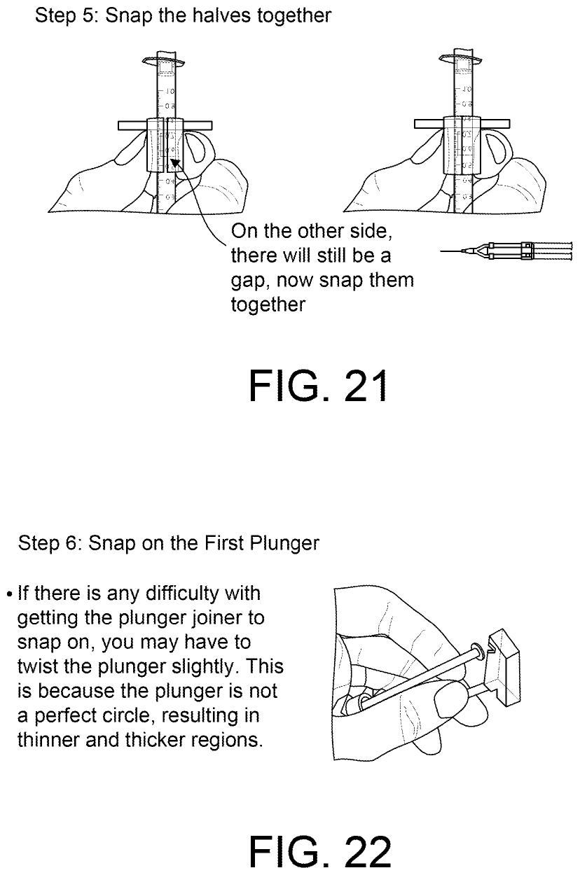

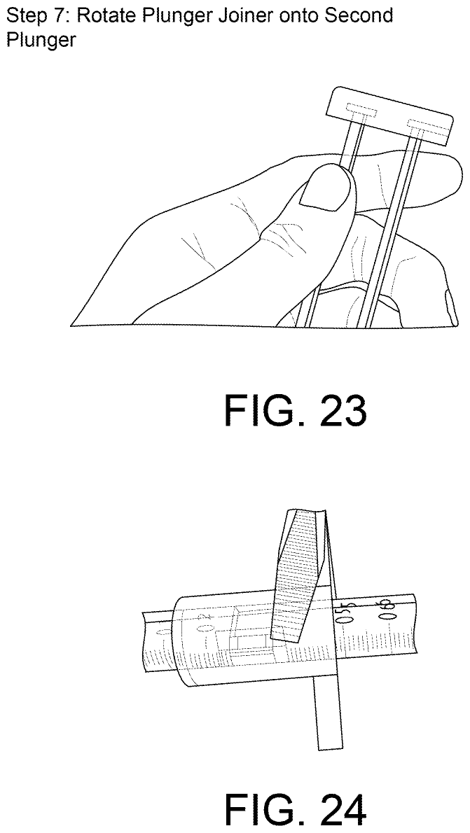

FIG. 11 and FIG. 12 show a disassembled embodiment of the example fluid delivery device 100. The syringe coupling 110 is made of two reversibly coupled pieces 110a and 110b that can be coupled together around the syringes 102a and 102b. FIG. 13 shows another disassembled embodiment of the example fluid delivery device 100. FIG. 14 shows an assembled device. FIG. 15 shows the syringe coupling 110 and the plunger coupling 112 in further detail. FIG. 16 shows a side view of an exemplary injection device. FIGS. 17-23 show the assembly of the fluid delivery device. FIG. 24 shows the removal of the syringe coupling 110 from the assembled fluid delivery device.

In another embodiment, the disassembled fluid delivery device can be contained within a kit. In some embodiments, at least part of the first syringe 102a and at least part of the first channel 204a have a first marking, and at least part of the second syringe 102b and at least part of the second channel 204b have a second marking that is distinct from the first marking for ease of assembly. In some embodiments, the kit is sterile. In some embodiments, the kit is not sterile.

In some embodiments, the kit further includes a portion of the first fluid and a portion of the second fluid. In some embodiments, each of the first fluid and/or the second fluid are sterile. In some embodiments, each of the first fluid and/or the second fluid are not sterile. In some embodiments, the syringes 102a and 102b are included in the kit containing the first fluid and/or the second fluid. In some embodiments, the syringes 102a and 102b can be filled with the first fluid and/or the second fluid. In some embodiments, the first syringe 102a contains the first fluid and the second syringe contains the second fluid.

In some embodiments, each of the first fluid and the second fluid are a liquid. In some embodiments, either one of the first fluid and/or the second fluid further includes a suspended solid. In some embodiments, the first fluid includes a first therapeutic agent and the second fluid includes a second therapeutic agent. As used herein, the term “therapeutic agent” refers to any chemical, molecule, moiety, compound, or combination thereof which can be used for the treatment of a disease. In some embodiments, either one of the first therapeutic agent and/or the second therapeutic agent is the liquid of the respective fluid. In some embodiments, either one of the first therapeutic agent and/or the second therapeutic agent is dissolved in the liquid of the respective fluid. In some embodiments, either one of the first therapeutic agent and/or the second therapeutic agent is the suspended solid of the respective fluid. In some embodiments, the first fluid can undergo a chemical reaction with the second fluid. In some embodiments, the first fluid is an acid and the second fluid is a base. In some embodiments, the first fluid is an oxidizing agent and the second fluid is a reducing agent. In some embodiments, the suspended solid undergoes the chemical reaction.

In another embodiment, provided is a method of manufacturing a fluid delivery device, the method comprising: additively manufacturing a channel guide separator 104; fabricating a first inner cannula 208a; and c) injecting a first sealant into the channel guide separator 104 around the first inner cannula 208a. In some embodiments, the method further comprises at least one sterilization step after any one of steps a), b), or c). In some embodiments, the at least one sterilization step includes autoclaving, chemical sterilization, sterilization by radiation, or other suitable sterilization methods. In some embodiments, step a) comprises injection molding, additive manufacturing, stereolithography printing, or other suitable manufacturing techniques. In some embodiments, at least one step is performed using an automated system.

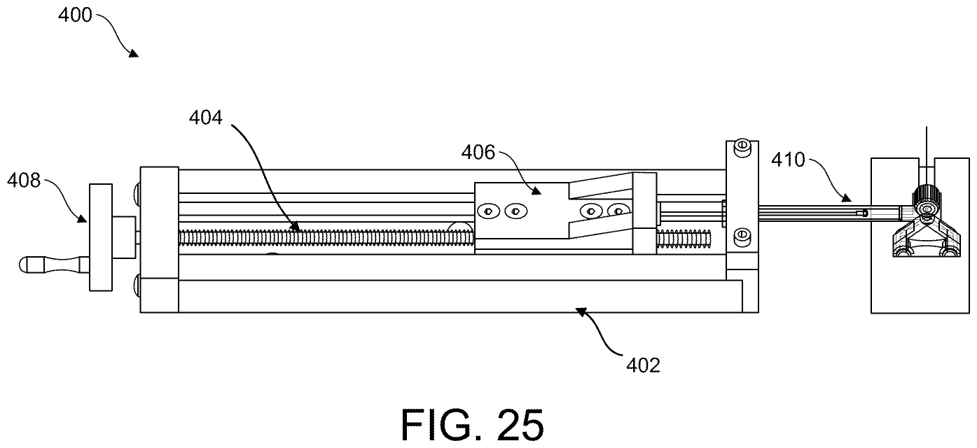

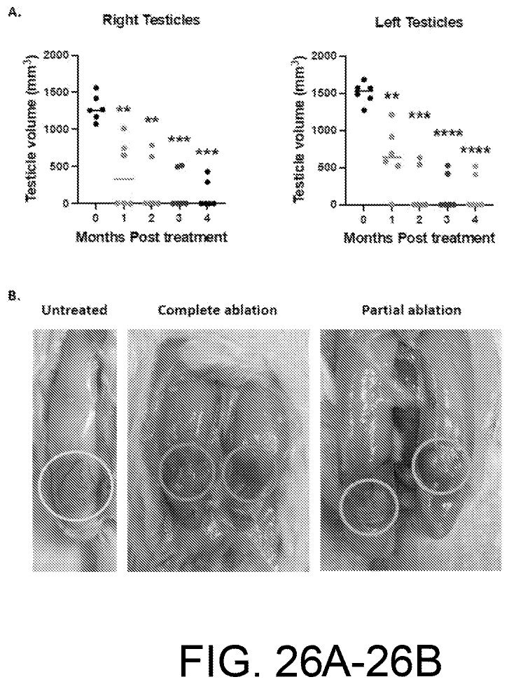

In some embodiments, step c) is performed with a linear rail assembly. In another embodiment, provided is a linear rail assembly for manufacturing a fluid delivery device, comprising: a base comprising a rail; a slide movably coupled to the rail; an injector comprising a body configured to receive a viscous fluid, an injection tip, and a plunger adjacent to the slide; and a torque mechanism for moving the slide along the rail; wherein a torque force required to operate the torque mechanism is less than a normal force required to depress the plunger. FIG. 25 shows an exemplary linear rail assembly 400. The base 402 contains the rail 404, on which the slide 206 is mounted. Turning the knob 408 provides a torque that drives the slide 406 along the rail 404. The slide 406 is adjacent to the plunger of the injector 410. As the slide 406 is driven down the rail 404, the plunger of the injector 410 is depressed, which injects the fluid contained within the body of the injector 410 into a fluid delivery device.

In some embodiments, the body of the injector contains a sealant. In some embodiments, the sealant is epoxy, polyester resin, polyurethane resin, acrylic resin, latex resin, silicone resin, or other suitable sealants or combinations thereof. In some embodiments, operating the torque mechanism injects the sealant into any of the disclosed fluid delivery devices.

In some embodiments, the torque mechanism is a rotatable knob. In some embodiments, the torque mechanism is turned by a motor.

In some embodiments, the torque mechanism further comprises a pitch of from about 0.005″ to about 0.2″ per revolution, including exemplary values of about 0.006″ per revolution, about 0.008″ per revolution, about 0.01″ per revolution, about 0.015″ per revolution, about 0.02″ per revolution, about 0.025″ per revolution, about 0.03″ per revolution, about 0.035″ per revolution, about 0.04″ per revolution, about 0.045″ per revolution, about 0.05″ per revolution, about 0.055″ per revolution, about 0.06″ per revolution, about 0.065″ per revolution, about 0.07″ per revolution, about 0.075″ per revolution, about 0.08″ per revolution, about 0.085″ per revolution, about 0.09″ per revolution, about 0.095″ per revolution, about 0.1″ per revolution, about 0.125″ per revolution, about 0.15″ per revolution, and about 0.175″ per revolution.

### METHOD OF USE

In another embodiment, provided is a method of simultaneously administering two or more fluids using any of the disclosed fluid delivery device. As used herein, the term “two or more fluids” refers to any two or more fluids which can have different chemical compositions, different concentrations, different physical properties, or which are the same but are contained within two or more separate vessels. In some embodiments, each of the first fluid and the second fluid are a liquid. In some embodiments, either one of the first fluid and/or the second fluid further includes a suspended solid. In some embodiments, the first fluid includes a first therapeutic agent and the second fluid includes a second therapeutic agent. In some embodiments, either one of the first therapeutic agent and/or the second therapeutic agent is the liquid of the respective fluid. In some embodiments, either one of the first therapeutic agent and/or the second therapeutic agent is dissolved in the liquid of the respective fluid. In some embodiments, either one of the first therapeutic agent and/or the second therapeutic agent is the suspended solid of the respective fluid. In some embodiments, the first fluid can undergo a chemical reaction with the second fluid. In some embodiments, the first fluid is an acid and the second fluid is a base. In some embodiments, the first fluid is an oxidizing agent and the second fluid is a reducing agent. In some embodiments, the suspended solid undergoes the chemical reaction.

In some embodiments, the chemical reaction can produce heat. In some embodiments, the heat is sufficient to ablate at least a portion of a tissue. In some embodiments, the tissue is a tumor tissue, a testicular tissue, an ovarian tissue, or a uterine tissue.

In another embodiment, provided is a method of treating a subject in need, the method comprising injecting two different fluids into the subject using any of the disclosed the fluid delivery devices. As used herein, the term “two different fluids” refers to any two fluids which can have different chemical compositions, different concentrations, different physical properties, or which are the same but are contained within two or more separate vessels. In some embodiments, each of the first fluid and the second fluid are a liquid. In some embodiments, either one of the first fluid and/or the second fluid further includes a suspended solid. In some embodiments, the first fluid includes a first therapeutic agent and the second fluid includes a second therapeutic agent. In some embodiments, either one of the first therapeutic agent and/or the second therapeutic agent is the liquid of the respective fluid. In some embodiments, either one of the first therapeutic agent and/or the second therapeutic agent is dissolved in the liquid of the respective fluid. In some embodiments, either one of the first therapeutic agent and/or the second therapeutic agent is the suspended solid of the respective fluid. In some embodiments, the first fluid can undergo a chemical reaction with the second fluid. In some embodiments, the first fluid is an acid and the second fluid is a base. In some embodiments, the first fluid is an oxidizing agent and the second fluid is a reducing agent. In some embodiments, the suspended solid undergoes the chemical reaction.

1. A fluid delivery device for delivering at least two fluids, the device comprising:
a first fluid pathway having a first upstream large volume and a first downstream small volume connected in fluid communication with the first upstream large volume, wherein a diameter of the pathway decreases from the first upstream large volume to the first downstream small volume,
the first fluid pathway further comprising a first exit having an exit diameter smaller than a storage diameter of the first upstream large volume;
a second fluid pathway having a second upstream large volume and a downstream second small volume connected in fluid communication with the second upstream large volume, wherein a diameter of the pathway decreases from the second upstream large volume to the downstream small volume,
the second fluid pathway further comprising a second exit having an exit diameter smaller than a storage diameter of the first upstream large volume; and
an insertion member containing the first and second exits in a closely proximate relationship and configured to insert the first and second exits through tissue to a distribution location without prior mixing of the fluids.

2. The fluid delivery device of claim 1,
wherein the first upstream large volume is a first syringe having a proximal end and a distal end, comprising a first plunger, and configured to receive a first fluid; and
wherein the second upstream large volume is a second syringe having a proximal end and a distal end, comprising a second plunger, and configured to receive a second fluid.

3. The fluid delivery device of claim 2, further comprising a syringe coupling around each of the distal end of the first syringe and the distal end of the second syringe and a plunger coupling connected to the first plunger of the first syringe and the second plunger of the second syringe.

4. (canceled)

5. The fluid delivery device of claim 1,
wherein the insertion member is a syringe tip comprising a coupling mechanism and a needle portion;
wherein the coupling mechanism connects the first fluid pathway and the second fluid pathway to the needle portion; and
wherein the needle portion comprises a main cannula.

6. The fluid delivery device of claim 1, further comprising a channel guide separator comprising a first channel, a second channel, and a central exit port;
wherein a proximal end of the insertion member is coupled to a distal end of the central exit port;
wherein the first fluid pathway comprises at least a part of the first channel;
wherein the second fluid pathway comprises at least a part of the second channel; and
wherein a distal end of the first channel and a distal end of the second channel merge into the central exit port.

7. (canceled)

8. The fluid delivery device of claim 6, wherein the first exit comprises a first inner cannula;
wherein a proximal end of the first inner cannula is in the first channel and a distal end of the first inner cannula is in a distal end of the insertion member; and
wherein the first inner cannula is contained by the first channel, the central exit port, and the insertion member.

9. The fluid delivery device of claim 8, further comprising a first sealant surrounding the first inner cannula, wherein the first sealant creates a barrier to the central exit port.

10. (canceled)

11. The fluid delivery device of claim 8,
wherein the second exit comprises the insertion member; and
wherein the first exit is encompassed by the second exit.

12. The fluid delivery device of claim 8, wherein the second exit comprises a second inner cannula;
wherein a proximal end of the second inner cannula is in the second channel and a distal end of the second inner cannula is in a distal end of the insertion member;
wherein the second inner cannula is contained by the second channel, the central exit port, and the insertion member; and
wherein the distal end of the first inner cannula is adjacent to the distal end of the second inner cannula.

13. The fluid delivery device of claim 12, further comprising a second sealant surrounding the second inner cannula, wherein the second sealant creates a barrier to the central exit port.

14-15. (canceled)

16. The fluid delivery device of claim 1,
wherein the first fluid pathway has a first volume and the second fluid pathway has a second volume; and
wherein the first volume is the same as the second volume.

17-20. (canceled)

21. A method of simultaneously administering two or more fluids using the fluid delivery device of claim 1.

22-27. (canceled)

28. A method of treating a subject in need, the method comprising injecting two different fluids into the subject using the fluid delivery device of claim 1.

29. The method of claim 28, wherein at least one of the fluids comprises a therapeutic agent.

30. The method of claim 28, wherein at least one of the fluids can undergo a chemical reaction with at least a second one of the fluids; and
wherein at least one of the fluids is an acid and at least a second one of the fluids is a base.

31. (canceled)

32. The method of claim 30, wherein the chemical reaction can produce heat sufficient to ablate at least a portion of a tissue.

33. (canceled)

34. The method of claim 32, wherein the tissue is a tumor tissue, a testicular tissue, an ovarian tissue, or a uterine tissue.

35-44. (canceled)

45. A method of manufacturing a fluid delivery device, the method comprising:
a) additively manufacturing a channel guide separator;
b) fabricating a first inner cannula; and
c) injecting a first sealant into the channel guide separator around the first inner cannula.

46-48. (canceled)

49. The method of claim 45, wherein step c) is performed with a linear rail assembly.

50. (canceled)

51. A linear rail assembly for manufacturing a fluid delivery device, comprising:
a base comprising a rail;
a slide movably coupled to the rail;
an injector comprising a body configured to receive a viscous fluid, an injection tip, and a plunger adjacent to the slide; and
a torque mechanism for moving the slide along the rail;
wherein a torque force required to operate the torque mechanism is less than a normal force required to depress the plunger.

52-56. (canceled)
